(19) United States
(12) Patent Application Publication
(10) Pub. No.: US 2025/0174318 A1
(43) Pub. Date: May 29, 2025

(54) DIGITAL MEDICINE COMPANION FOR CDK INHIBITOR MEDICATIONS FOR CANCER PATIENTS

(71) Applicant: PFIZER INC., New York, NY (US)

(72) Inventors: Anthony LAMBROU, New York, NY (US); Ofer WAKS, New York, NY (US); Caroline J. HOANG, Peapack, NJ (US); Xuemei CAI, Cambridge, MA (US); Ahsan HUDA, New York, NY (US); Timothy DOUGHERTY, Collegeville, PA (US); Dennis P. HANCOCK, Collegeville, PA (US); Joshua RAYSMAN, San Diego, CA (US); Solomon RAVICH, New York, NY (US)

(21) Appl. No.: 18/725,376

(22) PCT Filed: Dec. 27, 2022
(86) PCT No.: PCT/IB2022/062810

§ 371 Date: Jun. 28, 2024

Related U.S. Application Data
(60) Provisional application No. 63/295,109, filed on Dec. 30, 2021.

(51) Int. Cl. G16H 10/40; G16H 50/20; G16H 50/70

(52) U.S. Cl. CPC G16H 10/40; G16H 50/20; G16H 50/70

(57) ABSTRACT

The present disclosure relates to a digital medicine companion for patients undergoing oncology treatments. A patient may enter symptoms on a prescription software. A wearable device may passively collect other healthcare data such as biological data and/or physical activity data. The patient may therefore be monitored using the prescription software and/or wearables. Furthermore, the prescription software may be integrated with biofluid testing systems. For example, an at-home biofluid monitoring kit and/or a laboratory system may communicate with the prescription software and/or its backend server. The healthcare data collected through the monitoring and the biofluid testing may be fed into a machine learning model, which may output whether the patient is likely to develop side effects such as cytopenia. One or more alert notifications, e.g., to a clinician dashboard and/or to the prescription software, may be triggered when the machine learning model determines a higher likelihood of such side effects.

### RELATED APPLICATIONS

This application claims priority to U.S. Provisional Application No. 63/295,109 filed on Dec. 30, 2021, of which has been incorporated herein by reference in its entirety.

### FIELD

The subject matter presented herein is directed to systems and methods for monitoring breast cancer treatments. Specifically, systems and methods for monitoring side effects associated with the CDK inhibitor treatment for breast cancer.

### BACKGROUND

Cancers occur when cancerous cells grow rapidly and crowd out normal cells. For breast cancer patients, cyclin-dependent kinase (CDK) inhibitors, such as palbociclib (e.g., Pfizer's Ibrance), are prescribed to inhibit the growth of the cancerous cells. However, there may be side effects of CDK inhibitor medications. For example, there is be an ongoing risk of cytopenia, specifically neutropenia, for breast cancer patients undergoing CDK inhibitor treatments. Other classes of cancer treatments may include poly (ADP)-ribose polymerase (PARP) inhibitors. PARP inhibitors block the cancerous cells from repairing damaged DNAs (e.g., due to other cancer drugs) and therefore causing the cancerous cells to die. PARP inhibitors may have side effects such as cytopenia, especially thrombocytopenia and neutropenia. Other oncology treatment drugs also have side effects.

The conventional side effect management for oncology treatments (e.g., CDK inhibitor treatments, PARP inhibitor treatments, etc.), is inadequate. For instance, detection of cytopenia requires frequent biofluid testing, e.g., blood testing about 12 times a year. Frequent biofluid testing may not be readily available to patients, especially to remote area patients. Furthermore, the biofluid testing regimen may be inconvenient, requiring in person visits to a lab, and patients may not necessarily adhere to these inconvenient testing regimens. To compound the problem, conventional side effect management is based on the sporadic clinical encounters—with limited patient-clinician face time. The sporadic clinical encounters may therefore provide an incomplete picture of the patients' conditions, often based on biased information from the patients (e.g., due to recall bias). These limited encounters with faulty information sharing may not be adequate for early detection of oncology treatment side effects such as cytopenia. As an example, breast cancer patients may consistently feel fatigued and weak (symptoms of cytopenia), which the patients may interpret as being usual for a cancer patient, and may not escalate to the clinicians during the encounters. A lack of timely escalation of different symptoms to clinicians during the sporadic clinical encounters may not be amenable to proactive management of the side effects of the oncology treatments.

### SUMMARY

In some embodiments, the present disclosure relates to a digital medicine companion for patients undergoing oncology treatments (e.g., CDK inhibitor treatments, PARP inhibitor treatments). In an embodiment, a patient may be provided with prescription software such as a smartphone application. The prescription software may allow the patient to enter the symptoms, e.g., “feeling fatigued.” A wearable device may passively collect other healthcare data such as biological data (e.g., heart rate, temperature, etc.) and/or physical activity data (e.g., movement, exercise, etc.). The patient may therefore be continuously or near continuously monitored using the prescription software and/or wearables. Furthermore, the prescription software may be integrated with biofluid testing systems. For example, an at-home biofluid monitoring kit and/or a laboratory system may communicate with the prescription software and/or its backend server. The healthcare data collected through the monitoring and the biofluid testing may be fed into a machine learning model, which may output whether the patient is likely to develop side effects such as cytopenia. One or more alert notifications, e.g., to a clinician dashboard and/or to the prescription software, may be triggered when the machine learning model determines a higher likelihood of such side effects. This prediction and the corresponding notifications may allow a clinician to intervene proactively to manage and alleviate the side effects.

In an embodiment, a computer implemented method may be provided. The method may include retrieving first health data comprising symptoms of a patient undergoing a cyclin-dependent kinase (CDK) inhibitor treatment; retrieving second health data comprising analysis of a biofluid sample collected from the patient; deploying a machine learning model on the first health data and the second health data to predict whether the patient will develop a side effect associated with the CDK inhibitor treatment; and in response to the machine learning model predicting that the patient will likely develop a side effect, generating a message to be transmitted to a clinician dashboard to trigger a notification on the clinician dashboard.

In another embodiment, another computer implemented method may be provided. The method may include retrieving first health data comprising symptoms of a patient undergoing a cyclin-dependent kinase (CDK) inhibitor treatment; retrieving second health data comprising analysis of a biofluid sample collected from the patient; deploying a machine learning model on the first health data and the second health data to predict whether the patient will develop a side effect associated with the CDK inhibitor treatment; and in response to the machine learning model predicting that the patient will likely develop a side effect, generating a message to be transmitted to a healthcare application executing on a client device associated with the patient to trigger a notification on the healthcare application.

In yet another embodiment, a system is provided. The system may include one or more processors; and a non-transitory storage medium storing computer program instructions that when executed by the one or more processors cause the system to perform operations including retrieving first health data comprising symptoms of a patient undergoing a cyclin-dependent kinase (CDK) inhibitor treatment; retrieving second health data comprising analysis of a biofluid sample collected from the patient; deploying a machine learning model on the first health data and the second health data to predict whether the patient will develop a side effect associated with the CDK inhibitor treatment; and in response to the machine learning model predicting that the patient will likely develop a side effect, triggering one or more notifications.

### BRIEF DESCRIPTION OF DRAWINGS

Other objects and advantages of the present disclosure will become apparent to those skilled in the art upon reading the following detailed description of exemplary embodiments and appended claims, in conjunction with the accompanying drawings, in which like reference numerals have been used to designate like elements, and in which:

FIG. 1 shows a block diagram of an illustrative operating environment for employing one or more embodiments of this disclosure.

FIG. 2 shows a block diagram of an architecture of an illustrative computing device that may perform one or more functions, according to the several embodiments of this disclosure.

FIG. 3 shows a block diagram of an illustrative architecture of operating environment, wherein one or more embodiments disclosed herein may be employed.

FIG. 4 shows a flow diagram of an illustrative method of training a prediction model for predicting side effects of oncology treatments, according to the several embodiments of this disclosure.

FIG. 5 shows a flow diagram of an illustrative method of deploying a prediction model for predicting side effects of oncology treatments, according to the several embodiments of this disclosure.

The figures are for purposes of illustrating example embodiments, but it is understood that the present disclosure is not limited to the arrangements and instrumentality shown in the drawings. In the figures, identical reference numbers identify at least generally similar elements.

### DESCRIPTION

Embodiments disclosed herein may provide a digital medicine companion for cancer patients undergoing oncology treatments (e.g., CDK inhibitor treatments, PARP inhibitor treatments, etc.). The oncology treatments are generally associated with side effects such as cytopenia (e.g., neutropenia, thrombocytopenia), infections, etc. For predicting, detecting, and managing these symptoms, the digital medicine companion may include, for example, a prescription software, wearable sensors, an integration with biofluid (e.g., blood, urine, saliva, nasopharyngeal fluid, etc.) testing systems (e.g., bloodwork systems), and back end analysis modules.

The prescription software may be provided in form of a smartphone application that may allow a patient to enter symptoms. The patient entered symptoms may include, for example, how the patient is feeling (e.g., fatigued), whether the patient is feverish, the patient's appetite level, and/or any other type of biological and psychological symptoms. The wearable sensors may passively collect data (e.g., without the involvement of the patient), such as movement data (e.g., activity level), and/or biological data (heartrate, blood oxygen saturation level). The integration with the biofluids testing systems may allow retrieval of biofluid analysis (e.g., bloodwork) data from any type of biofluid test (e.g., an at-home test kit, a lab test). The back end analysis modules may use one or more machine learning modules to predict whether the patient is likely to develop a side effect based on the patient entered data in the prescription software, the passively collected data from the wearables, and/or the biofluid analysis data from the integrated biofluid testing systems. If the machine learning modules predict a higher likelihood of the patient developing the side effects, one or more notifications to a clinician dashboard and/or the prescription software may be generated. The one or more notifications may allow for a timely clinical intervention and therefore a proactive management of the side effects.

It should be understood that below embodiments describing a digital medicine companion for oncology treatments are merely examples and should not be considered limiting. The embodiments may be equally applicable to managing side effects associated with different treatment regimens of non-oncology diseases. Furthermore, the side effects described below (e.g., cytopenia) are also just examples and the embodiments may be equally applicable to other non-listed side effects as well.

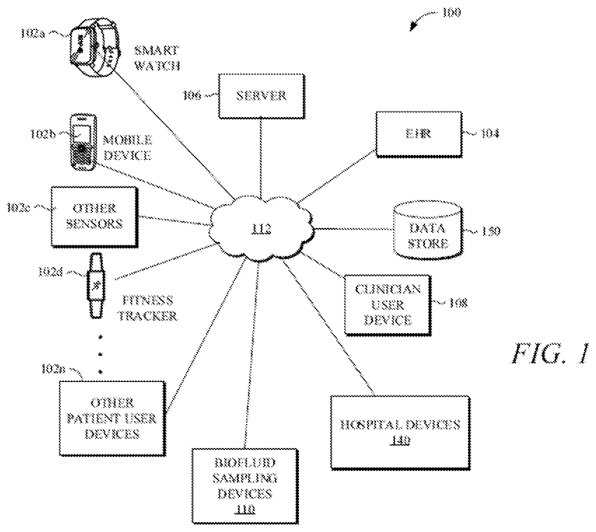

FIG. 1 shows a block diagram of an illustrative operating environment 100 for employing one or more embodiments of this disclosure. It should be understood that this and other arrangements described herein are set forth only as examples. Other arrangements and elements (e.g., machines, interfaces, functions, orders, and groupings of functions) can be used in addition to, or instead of, those shown in FIG. 1 as well as other figures, and some elements may be omitted altogether for the sake of clarity. Further, many of the elements described herein are functional entities that may be implemented as discrete or distributed components or in conjunction with other components, and in any suitable combination and location. Various functions or operations described herein are being performed by one or more entities including a hardware, firmware, software, and a combination thereof. For instance, some functions may be carried out by a processor executing instructions stored in memory.

As shown, the operating environment 100 may include patient facing user devices 102a-102n (collectively referred to as devices 102 or commonly referred to as device 102), biofluid sampling devices 110, a server 106, an electronic health record (EHR) system 104, network 112, hospital devices 140, a data store 150, and a clinician user device 108. It should also be understood that the singular or plural description of the devices are just for the sake of clarity in explanation and should not be considered limiting. For instance, the server 106 may include multiple servers and the clinician user device 108 may include multiple user devices. The different devices in the operating environment 100 may be interconnected through the network 112.

The patient facing user devices 102 may include any type of computing and/or sensing device that the patients may interact with. The non-limiting examples shown in the operating environment 100 may include a smartwatch 102a, a mobile device 102b (e.g., a smartphone), other smart sensors 102c (e.g., a smart ring, invisible sensors such as motion sensors, etc.), a fitness tracker 102d, and other patient facing user devices 102n (e.g., tablets, laptop computers, desktop computers, smart speakers, smart home systems, etc.). The patient facing user devices 102 may either passively collect or actively prompt a patient to enter data associated with management of side effects associated with oncology treatments (e.g., CDK inhibitors such as Pfizer's Ibrance, PARP inhibitors, etc).

For example, the smartwatch 102a may have multiple sensors to passively collect data from the patient. The multiple sensors may include accelerometers, gyroscopes, and/or other types of motion sensors that may track the physical activity of the patient. The smartwatch 102a may further include sensors to detect biological parameters such as body temperature, heart rate, blood glucose level, blood oxygen saturation level, and/or any other type of biological parameters. The biological parameters may be continuously and/or periodically collected by the smartwatch 102a—e.g., without the patient explicitly involved in the collection—and provided to other components (e.g., server 106) in the operating environment.

The mobile device 102b may be used by the patient for actively entering health related data. For example, the mobile device 102d may be a smartphone that may have a healthcare application, e.g., an electronic patient reported outcome (ePRO) application, installed therein. The healthcare application may prompt the patient to enter health related data. The health related data may include, for example, symptoms that the patient may be experiencing. For instance, the symptoms may include, “Fatigued,” “Weak,” “Feeling Fine,” etc. As another example, the patients may enter the symptoms in response to push notifications generated within the operating environment 100. For instance, the healthcare application may generate a push notification for the patient to enter data as to how the patient is feeling at the given point in time. The prompt may be, “How are you feeling this morning?” and the patient may enter, “I am feeling great.” The healthcare application may also display alert notification, which may be generated when the operating environment 100 determines that there is a likely risk of cytopenia, neutropenia, infections, etc. Another type of alert notification displayed by the healthcare application may be when the current disease behavior of the patient deviates significantly from the established baseline behavior. In response, the healthcare application may further allow the patient to communicate, synchronously or asynchronously, with the clinician.

The other sensors 102c may include devices such as smart rings, skin patches, ingestible sensors, and/or any other type of body attached or non-body attached sensors (generally referred to as invisibles or invisible devices/sensors). The other sensors 102c may detect biological or non-biological data. For instance, other sensors 102c may include a smart fabric measuring a body temperature of the patient. As another example, the other sensors 102c may include a smart home sensor measuring home temperature and/or humidity. As yet another example, the other sensors 102c may include a motion sensor that may detect/measure movement within a room. The other patient devices 102n may include any other type of device associated with the patients. For instance, the other patient devices 102n may include tablet computers, laptop computers, desktop computers, and/or any other computing devices associated with the patients and connected to the network 112.

The biofluid sampling devices 110 may include any kind of biofluid (also referred to as bodily fluids) monitoring device and/or service that may collect and analyze biofluid samples of the patient. The biofluids may include, for example, blood, saliva, urine, nasopharyngeal fluids, etc. For example, the biofluid sampling devices 110 may include a home mailed sample collection kit that may allow the patient to draw blood and/or collect another biofluid and send the kit back to lab for analysis. In other instances, the biofluid sampling devices 110 may include home mailed sample collection kit that may perform at least a portion of the analysis itself and provides the result of the analysis at one or more of the patient facing devices 102 and/or the server 106. The biofluid sampling devices 110 should further be understood to include monitoring devices used by clinicians in a lab setting or in a home visit setting. Therefore, any type of technology and/or service used for collecting patient biofluid samples and analyzing the samples for biological parameters should be considered within the scope of this disclosure. An example of the biological parameter measured by the biofluid sampling devices 110 may include white blood cell count in blood, wherein a lower white blood cell count may indicate an onset of cytopenia.

The network 112 may include any kind of communication network. For instance, the network 112 may include packet switching network supporting protocols such as TCP/IP. The network 112 may also include circuit switching networks supporting both wired and wireless telephony. The network 112 therefore may include components such as wires, wireless transmitters, wireless receivers, signal repeaters, signal amplifiers, switches, routers, communication satellites, and/or any other type of network and communication devices. Some non-limiting examples of the network 112 may include local area network (LAN), metropolitan area network (MAN), wide area network (WAN) such as the Internet, etc. These are just but a few examples of the network 112, and any kind of communication linkage between the different components of the operating environment 100 are to be considered within the scope of this disclosure.

The server 106 may include any type of computing devices that may provide the analytical functionality of training and deploying one or more machine learning models and/or establishing and deploying statistical analytic models. For instance, the server 106 may train a prediction model for predicting a side effect, e.g., whether a patient under an oncology treatment regimen (e.g., CDK inhibitor treatment regimen) will develop a side effect. The prediction model may be trained using a supervised training approach, with labeled ground truth data. The prediction model may include, for example, regression model, a gradient boosted regression model, a logistic regression model, a random forest regression model, an ensemble model, a classification model, a deep learning neural network, a recurrent neural network for deep learning, or a convolutional neural network for deep learning.

The machine learning based prediction models described above are just examples and other statistical models are to be considered within the scope of this disclosure. For instance, the server 106 may also establish an analytical model based on the continuously collected longitudinal healthcare data, wherein the analytical model may indicate a baseline healthcare behavior. When new healthcare data is received, the server 106 may compare the received data with the analytical model (e.g., against the baseline healthcare behavior) to determine whether the new healthcare data shows a significant deviation from the baseline health behavior. The server 106 may also generate one or more alert notifications, e.g., to patients and/or clinicians, indicating that the patient is likely to develop one or more adverse conditions (e.g., cytopenia, neutropenia, etc.) and/or that the patient's health behavior has significantly deviated from the baseline behavior.

The electronic health record (EHR) 104 may store the health records of the patients. The health records may include, for example, the patients' ongoing condition (e.g., breast cancer), prescribed medications, summaries of clinical encounters, and/or any other healthcare related data associated with the patients. In some embodiments, the EHR 104 may be maintained by healthcare providing entity (e.g., a hospital system).

The data store 150 may include any kind of database storing data collected from various sources within the operating environment 100. For instance, the data store 150 may store data collected, both passively and actively, from the patient facing devices 102. The data store 150 may also store data collected from biofluid sampling devices 110. Additionally, the data store 150 may store data sourced from EHR 104. Therefore, the data source 150 should be understood to store any kind of data in the operating environment 100.

The clinician user device 108 may be any kind of computing device showing a clinician dashboard. Non-limiting examples of the clinician user device 108 may include a mobile phone (e.g., a smartphone), a tablet computer, a laptop computer, a desktop computer, and/or any other type of computing device. The clinician dashboard may show information (e.g., demographic information, location information) and/or one or more alerts associated with the various patients.

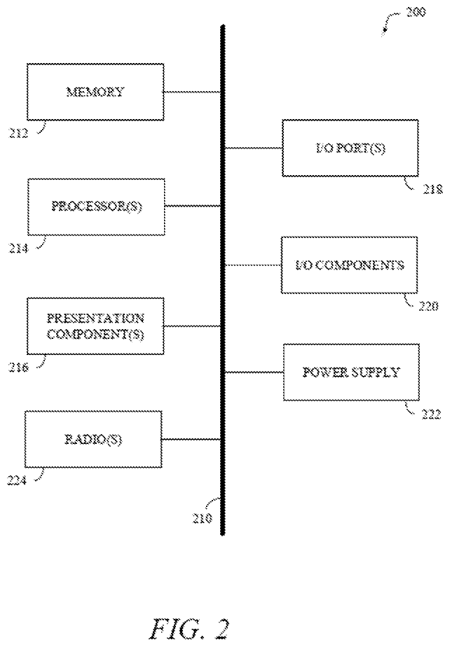

FIG. 2 shows a block diagram of an illustrative computing device 200 that may perform one or more functions described herein, according to the several embodiments of this disclosure. The computing device 200 is but one example of a suitable computing environment and is not intended to suggest any limitation as to the scope of use or functionality of embodiments of the disclosure. Neither should the computing device 200 be interpreted as having any dependency or requirement relating to any one or combination of components illustrated.

Embodiments of the disclosure may be described in the general context of computer code or machine-useable instructions, including computer-useable or computer-executable instructions, such as program modules, being executed by a computer or other machine, such as a personal data assistant, a smartphone, a tablet PC, or other handheld or wearable device, such as a smartwatch. Generally, program modules, including routines, programs, objects, components, data structures, and the like, refer to code that may perform particular tasks or implement particular data types. Embodiments of the disclosure may be practiced in a variety of system configurations, including handheld devices, consumer electronics, general-purpose computers, or more specialty computing devices. Embodiments of the disclosure may also be practiced in distributed computing environments where tasks are performed by remote-processing devices that are linked through a communications network. In a distributed computing environment, program modules may be located in both local and remote computer storage media including memory storage devices.

The computing device 200 may include a bus 210 that may directly or indirectly couple the following example devices: memory 212, one or more processors 214, one or more presentation components 216, one or more input/output (I/O) ports 218, one or more I/O components 220, and a power supply 222. Some embodiments of computing device 200 may further include one or more radios 224. Bus 210 represents what may be one or more busses (such as an address bus, data bus, or combination thereof). Although the various blocks of FIG. 2 are shown with lines for the sake of clarity, these blocks may represent logical, and not necessarily actual, components. For example, one may consider a presentation component 216 such as a display device to be an I/O component. Also, processors 214 may have their memories. Furthermore, distinction is not made between such categories as “workstation,” “server,” “laptop,” or “handheld device,” as all are contemplated within the scope of FIG. 2 and with reference to “computing device.”

The computing device 200 may include a variety of computer-readable media. Computer-readable media can be any available media that can be accessed by computing device 200 and may include both volatile and nonvolatile, removable and non-removable media. By way of example, and not limitation, computer-readable media may comprise computer storage media and communication media. Computer storage media may include both volatile and nonvolatile, removable and non-removable media implemented in any method or technology for storage of information such as computer-readable instructions, data structures, program modules, or other data. Some non-limiting examples of computer readable media may include RAM, ROM, EEPROM, flash memory or other memory technology, CD-ROM, digital versatile disks (DVDs) or other optical disk storage, magnetic cassettes, magnetic tape, magnetic disk storage or other magnetic storage devices, or any other medium which can be used to store the desired information and which can be accessed by computing device 200. Communication media may embody computer-readable instructions, data structures, program modules, or other data in a modulated data signal such as a carrier wave or other transport mechanism and includes any information delivery media. The term “modulated data signal” may refer to a signal that has one or more of its characteristics set or changed in such a manner as to encode information in the signal. By way of example, and not limitation, communication media may include wired media, such as a wired network or direct-wired connection, and wireless media, such as acoustic, RF, infrared, and other wireless media. Combinations of any of the above should also be included within the scope of computer-readable media.

The memory 212 may include computer storage media in the form of volatile and/or nonvolatile memory. The memory 212 may be removable, non-removable, or a combination thereof. Some non-limiting examples of hardware devices for the memory 212 include solid-state memory, hard drives, optical-disc drives, etc.

The computing device 200 may include one or more processors 214 that read data from various entities such as memory 212 or the I/O components 220. The presentation component(s) 216 may present data indications to a user or other device. Exemplary presentation components may include a display device, speaker, printing component, and the like.

The I/O ports 218 may allow computing device 200 to be logically coupled to other devices, including I/O components 220, some of which may be built in. Non-limiting examples of the I/O components 220 may include a microphone, joystick, game pad, satellite dish, scanner, printer, or a wireless device. The I/O components 220 may provide a natural user interface (NUI) that processes air gestures, voice, or other physiological inputs generated by a user. In some instances, inputs may be transmitted to an appropriate network element for further processing. A NUI may implement any combination of speech recognition, touch and stylus recognition, facial recognition, biometric recognition, gesture recognition both on screen and adjacent to the screen, air gestures, head and eye tracking, and touch recognition associated with displays on the computing device 200. The computing device 200 may be equipped with cameras, such as stereoscopic camera systems, infrared camera systems, RGB camera systems, and combinations of these, for gesture detection and recognition. Additionally, the computing device 200 may be equipped with accelerometers or gyroscopes that enable detection of motion.

Some embodiments of computing device 200 may include one or more radio(s) 224 (or similar wireless communication components). The radio may transmit and receive radio or wireless communications. The computing device 200 may be a wireless terminal adapted to receive communications and media over various wireless networks. Computing device 200 may communicate via wireless protocols, such as code division multiple access (“CDMA”), global system for mobiles (“GSM”), or time division multiple access (“TDMA”), as well as others, to communicate with other devices. The radio communications may be a short-range connection, a long-range connection, or a combination of both a short-range and a long-range wireless telecommunications connection.

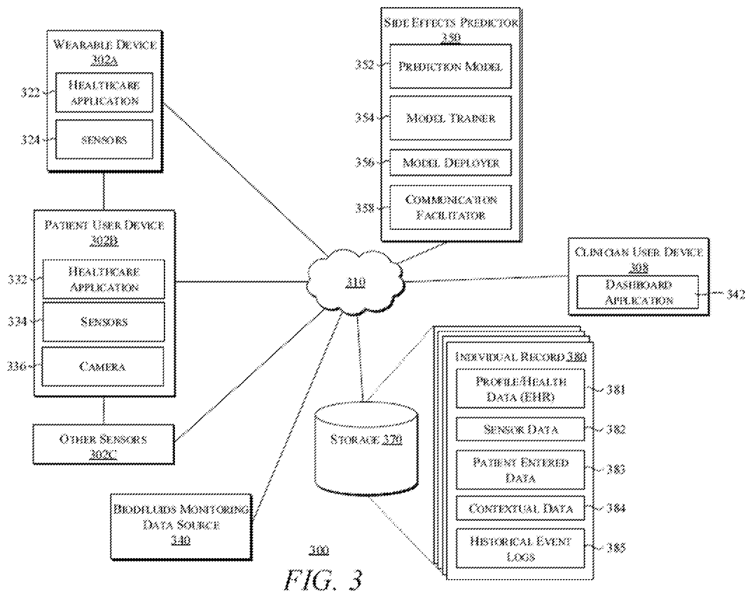

FIG. 3 shows a block diagram of an illustrative architecture of operating environment 300, wherein one or more embodiments disclosed herein may be employed. The shown architecture may be implemented by one of more components/devices (e.g. computing device 200 shown in FIG. 2) of the operating environment 100 shown in FIG. 1. It should however be understood that the shown architecture is just but an example and architectures with additional, alternative, or fewer number of components should also be considered within the scope of this disclosure. It should further be understood that components shown as a single components or plural components are also just examples: a single component may include multiple iterations of the same component or multiple constituent sub-components and the functionality of plural components may be achieved by a single component.

The operating environment 300 may be used for a continuous or near-continuous digital monitoring of patients undergoing oncology treatment regimens such as CDK inhibitor treatment regimens (e.g., Pfizer's Ibrance), PARP inhibitor treatment regimens, etc. and triggering alert notifications to the patients and/or clinicians as needed. Furthermore, the operating environment 300 may provide a seamless and continuous connectivity between the patients and clinicians. This continuous or near-continuous monitoring may allow for a proactive management of the side effects associated with the oncology treatment medications-a machine learning model may predict onset of one or more conditions and therefore may facilitate early clinical interventions against those conditions. The operating environment 300 may further have a seamless integration of biofluids monitoring data source 340 such that biofluid analysis (e.g., bloodwork) may be continuously tracked to detect/predict cytopenia. To implement these and other functions, the operating environment 300 may include patient facing devices (e.g., wearable device 302a, patient user device 302b, other sensors 302c, etc.) to gather healthcare data and other data from the patients, to provide the alert notification to the patients and/or clinicians, and to facilitate communication between clinicians and patients. The data gathered from the patient facing devices 302 and biofluids monitoring data source 340 may be stored in the storage 370 (e.g., as individual records 380). The analysis components (e.g., side effect predictor 350) may use the stored data and other data to predict and/or detect the side effects associated with oncology treatment medications. Based on the analysis, alert notifications may be sent to clinicians (e.g., through a clinician user device 308). The components of the operating environment 300 may be interconnected through a network 310.

With regard to the patient facing devices 302, these devices may include, for example, the wearable device 302a, the patient user device 302b, and other sensors 302c. The wearable device 302a may include any kind of wearable device: non-limiting examples include a smartwatch, a fitness tracker, a smart ring, etc. In some embodiments, the wearable device 302a may include a healthcare application 322. The wearable device 302a or any application installed thereon (e.g., healthcare application 322) may be a medically prescribed component within the operating environment 300. The healthcare application 322 may be a computer program installed on the wearable device 302a to collect healthcare data, perform pre-processing of the collected data in some instances, and transmit the data to the patient user device 302b or to a remote server (e.g., a server implementing side effects predictor 350 and/or storage 370). Particularly, the healthcare application 322 may interface with the operating system of the wearable device (e.g., through API calls) to gather the data from the sensors 324.

The sensors 324 may include any type of sensors that may continuously or periodically gather data from the patient wearing the wearable device 302a. For example, the sensors 324 may include biological sensors, such as a temperature sensor to measure the body temperature (it should be understood that the temperature sensor may be non-biological and may measure the ambient temperature), heart rate monitor, an electrocardiogram sensor to collect electrocardiogram data when prompted by the patient, a glucose monitor, a sweat monitor, blood oxygen saturation level monitor, blood pressure monitor, and/or any other type biological sensors.

The sensors 324 may also include accelerometers to determine directional movement, gyroscopes to detect the orientation, and/or any other type of sensors gather positional or movement data. These sensors 324 may be triggered by the healthcare application 322 (e.g., through API calls to the operating system of the wearable device 302a) to collect the corresponding data. Alternatively, the wearable device 302a may not have the healthcare application 322 and the triggering may be received from the patient user device 302b (e.g., from its healthcare application 332) or remotely through the network 310. In other embodiments, the wearable device 302a may itself be continuously or periodically activating the sensors 324 and may pass the collected sensor data along to the healthcare application 324 (and/or healthcare application 332 or a remote device connected via the network 310). The biological data and the movement data collected by the sensors 324 may be collectively or commonly referred to as healthcare data (or health data).

In other words, the sensors 324 may be collecting healthcare data passively, i.e., without an active involvement of the patient. For instance, the sensors 324 may be monitoring the patient's biological data and/or physical movements because the sensors 324 are within the wearable device 302a and therefore are continuously attached to the patient. This passive collection of the healthcare data does not require the patient's continuous attention of the patient and is therefore less burdensome.

The patient user device 302b may include any kind of computing device used by the patient. For example, the patient user device 302b may include a mobile phone such as a smartphone, a tablet device, a laptop computer, a desktop computer, and/or any other type of computing device. A healthcare application 332 (e.g., an ePRO) may be installed on the patient user device 302b. The healthcare application 332 should be understood to include a standalone application (e.g., a smartphone app) or a web-based application (e.g., accessed using a browser). It should further be understood that the healthcare application 332 may also be medically prescribed. The healthcare application 332 may provide an interface (e.g., a graphical user interface) for the patient to view alert notifications, communicate with a clinician, and/or actively enter healthcare data (e.g., observed symptoms).

As an example, the healthcare application 332 may be used gather further information on a prediction based on the data collected by the sensors 324 of the wearable device. For instance, using the passively collected data, the side effects predictor 350 may predict an onset of a side effect of oncology treatments (e.g., CDK inhibitors, PARP inhibitors, etc.). For example, the sensors 324 of the wearable device 302a may measure less body movement compared to an established baseline; and side effects predictor 350 may predict that the patient may be experiencing fatigue. In response to this prediction, an alert notification (e.g., by communication facilitator 358) may be sent to the healthcare application 332. The alert notification may be, for example, “We have detected lower activity level? Are you feeling fatigued?” and prompt the patient to respond. For the response, the healthcare application 332 may provide choices such as “Extremely Fatigued,” “Moderately Fatigued,” or “Not Feeling Fatigued.” As another example, the sensors 324 of the wearable device 302a may measure a lower than usual heart rate. In response to this determination, the server may transmit another alert notification to the healthcare application 332. The alert notification may include, for example, “We have detected a lower heart rate. Do you feel lightheaded?” and prompt the patient to respond. For the response, the healthcare application 332 may provide choices such as “Extremely Lightheaded,” “Moderately Lightheaded,” or “No Lightheadedness.” This are just but a few examples of the alert notifications to the patient and other types of alert notification should also be considered within the scope of this disclosure.

In addition to the prompts corresponding to alert notifications, the healthcare application 332 may request the user to periodically (e.g., without a trigger for data entry) enter healthcare data. For instance, the healthcare application 332 may prompt the patient to enter the observed symptoms every morning, daytime, and evening. Other non-limiting examples of the actively entered data include body temperature (if not collected by the sensors 324 of the wearable device 302a), bowel movement, level of pain experienced, stress level, anxiety level, the time of intake of a prescription medication, exercise activity (if not captured by the wearable device 302a), and/or any other type of healthcare data.

In addition to the alert notifications, the healthcare application 332 may also provide two-way connectivity between the patient and the clinician. The two-way connectivity between the patient and the clinician may be facilitated by the communication facilitator 358 of the side effects predictor 350. The two-way connectivity may allow the patient to establish any type of synchronous (e.g., voice or a video call) or asynchronous (e.g., message exchanges) with the clinician. The two-way connectivity may be initiated in response to an alert notification. For instance, the side effects predictor 350 may predict an onset of cytopenia and the communication facilitator 358 may have generated an alert provided on the healthcare application 332. When the patient selects the alert notification (e.g., a notification badge), an interface may be provided by the healthcare application 332 for the patient to initiate a communication session with the clinician (e.g., messaging or calling the clinician). For instance, the patient may pose questions through the healthcare application 332 itself and the clinician's response may be displayed within the healthcare application 332. The two-way connectivity may also be leveraged to provide educational materials to the patient. The educational material in some embodiments may include cognitive behavior therapy (CBT) based behavior modification encouragement materials. These materials may be provided to the patient based on the healthcare data passively collected by the wearable device 302a, actively collected by the patient user device 302b, and analyzed by the side effects predictor 350. The CBT-based materials may be in the form of audio, video, and/or text and encourage the patient to make healthier choices on food (e.g., BRAT (Bananas, Rice, Applesauce, Toast) diet, rest and exercise, stress management, and/or any other metric associated with maintaining a good quality of life while under the oncology treatment regimen.

It should however be understood that the connectivity through the healthcare application 332 is just an example, and other types of connectivity should also be considered within the scope of this disclosure. For instance, the two-way connectivity may be established through other components other than the healthcare application 332. For example, the patient may make a phone call to the doctor, send an e-mail, send a message, etc. without necessarily using the healthcare application for these types of communication.

The sensors 334 in the patient user device 302b may include any type of sensors such as heart rate sensors (e.g., when the patient brings the patient user device 302b closer to the body), glucose monitors (e.g., using an infrared camera), accelerometers, gyroscopes, etc. Generally, any type of biological and/or movement sensor should be considered within the scope of this disclosure. For example, in case of the patient user device 302b being a mobile device (e.g., a smartphone), the patient user device 302b too may monitor the user's movement using the sensors 334. The sensors 334 may detect the number of steps taken by the patient throughout the day and/or other activities (e.g., exercise) performed by the patient throughout the day. In other words, the sensors 334 too may be used to passively collect movement data of the patient. In some embodiments, the sensors 334 may enable an active data collection. For instance, the sensors 334 may include an infrared camera and the patient user device 302b may prompt the patient to hold their finger against the camera to detect biological attributes such as blood glucose level, blood oxygen saturation level, etc. In another example, the sensors 334 may include a heart rate sensor, which when brought close to the patient's body, may measure the patient's heart rate.

The camera 336 may include an optical camera that the patient may use to take healthcare related pictures. The picture may be of a relevant body part, e.g., picture of the patient's hand showing the state of the skin. The pictures may be sent to the storage 370 and/or provided to the clinician. The clinician may use the pictures for diagnostic purposes (e.g., to determine whether a particular side effect is improving or worsening) and/or for therapeutic purposes (e.g., to determine whether to adjust the dosage the medication the patient is taking.)

The other sensors 302c may be any kind of sensors measuring one or more biological or physical attributes of the patient. An example of the other sensors 302c may be an ingestible sensor that may be measure the effect on gut activity of the oncology treatment regimen and/or other associated prescription medications. Another example may be a patch sensor that may be attached to the skin to measure attributes such skin temperature and/or the movement of the body part that the patch sensor is attached to. The sensors 302c may further include a blood pressure monitor that may communicate measurements to the patient user device 302b or any other device within the operating environment 300. Other examples of the sensors 302c may include smart fabrics, smart belts, sub-cutaneous sensors, etc. These are just but a few examples of the sensors and any type of body-worn or non-body-worn sensor should be considered within the scope of this disclosure. The non-body worn sensors may be referred to as invisibles or invisible sensors/devices.

The biofluids monitoring data source 340 may be any kind of biofluid (e.g., blood, urine, saliva, nasopharyngeal fluid) analysis device or system. For example, the biofluids monitoring data source 340 may include at home sample collection kit. The at home sample collection kit may be mailed to the patient's home and used by the patient to collect the sample to be sent to a lab. The lab may in turn perform the biofluid analysis (e.g., determine white blood cell count in a blood sample) and provide the analysis data through the network 310 to other components (e.g., storage 370) of the operating environment 300. In other embodiments, the home collection kit may have some sample analysis capacity in combination of the patient user device 302b—e.g., an analysis component in the kit may connect with the patient user device 302b to provide the measured data.

The biofluids monitoring data source 340 may further include other types of sources such as labs, doctor's offices, and/or any type of equipment and/or establishment for collecting and analyzing biofluid samples. Regardless of its type, the biofluids monitoring data source 340 may provide the measured data to other components within the operating environment 300 (e.g., through the network 310).

As described above, the data collected—either passively or actively—by one or more of the wearable device 302a, patient user device 302b, other sensors 302c, and the biofluids monitoring data source 340 may be received by other components in the operating environment through the network 310. The network 310 may include any kind of communication network. For instance, the network 310 may include packet switching network supporting protocols such as TCP/IP. The network 310 may also include circuit switching networks supporting both wired and wireless telephony. The network 310 therefore may include components such as wires, wireless transmitters, wireless receivers, signal repeaters, signal amplifiers, switches, routers, communication satellites, and/or any other type of network and communication devices. Some non-limiting examples of the network 310 may include local area network (LAN), metropolitan area network (MAN), wide area network (WAN) such as the Internet, etc. These are just but a few examples, and any kind of communication linkage between the different components of the operating environment are to be considered within the scope of this disclosure.

The data received from the patient facing devices (e.g., wearable device 302a, patient user device 302b, other sensors 302c, etc.) and biofluids monitoring data source 340 may be stored in the storage 370. The storage 370 may include any kind of storage technology such as hard drive storage, solid state storage, data server storage, etc. Although a single storage 370 is shown for the clarity of explanation, the storage 370 should be understood to include multiple, geographically distributed components. For example, the storage 370 may be distributed among multiple data centers and incorporate multiple levels of redundancies.

In some embodiments, the storage 370 may store individual records 380 containing the data for the corresponding patients. In other words, an individual record 380 may be associated with the patient. It should however be understood that this individual record 380—based organization of data is just an example and should not be considered limiting. Any kind of data organization (e.g., relational, object oriented) should be considered within the scope of this disclosure.

As shown, an individual record 380 of a patient may include a profile/health data (e.g., electronic health record (EHR) data) 381, sensor data 382, patient entered data 383, contextual data 384, and historical event logs 385. However, these are just some examples of the pieces of data types within the individual record 380; and additional, alternative, or fewer pieces of data types should also be considered within the scope of this disclosure. Furthermore, the discrete data types shown herein are just examples as well, and a data type may include aspects of other data types. For example, the profile/health data 381 may incorporate historical event logs 385.

The profile/health data 381 may include the electronic health record of the corresponding patient. The profile/health data 381 may therefore include a demographic information, comprehensive medical history, family medical history, allergies, ongoing conditions, records of clinical encounters, other notes from clinicians, prescription medications, laboratory results such as bloodwork results, and/or any other type of healthcare data for the patient. For example, the profile/healthcare data 381 may include information about the treatment regime using a CDK inhibitor and any observed side effects of administrating the CDK inhibitor. As another example, the profile/healthcare data 381 may include information about the treatment regime using a PARP inhibitor and any observed side effects of administrating the PARP inhibitor. In some embodiments, the profile/health data 381 may be sourced to the storage 370 from other entities. For instance, the profile/health data 381 may be managed by a healthcare providing entity (e.g., a hospital), and the operating environment 300 may retrieve the data from the healthcare providing entity.

The sensor data 382 may be the data from the patient facing sensors such as the sensors 324 of the wearable device 302a, sensors 334 of the patient user device 302b, and/or other sensors 302c. The sensor data 382 may therefore include data from biological sensors (e.g., heart rate monitors, blood pulse oximeters), the movement sensors (e.g., accelerometers and/or gyroscopes), and/or any other type of sensors. The sensor data 382 may be stored in association with the timestamps of when the data was collected. The timestamps may allow the operating environment 300 to detect the patient's activity and condition throughout the day. As used herein, the sensor data 382 should be generally understood to include any kind of passively collected data (e.g., movement passively detected by a wearable), or data captured by the patient actively engaging with the sensor (e.g., the patient putting their finger on an infrared camera to measure various biological attributes).

The patient entered data 383 may include any kind of data actively entered by the patient (e.g., through the healthcare application 332). The patient entered data 383 may therefore include, the patient's entry of their symptoms (e.g., “Fatigued,” “Depressed,” etc.) at a particular point in time. As another example, the patient entered data 383 may include the patient's response to various alert notifications provided by the healthcare application 332. The patient entered data 383 may further include other biological data not captured by the sensors (e.g., sensors 324, 334, and/or 302c). For instance, such biological data may include blood glucose level, blood oxygen saturation level, blood pressure, etc. captured by devices (e.g., an external blood pressure monitor) operating with active user engagement within the operating environment 300. As with the sensor data 382, the patient entered data 383 may also be organized using the timestamps. In other words, the timestamps may be used to correlate the sensor data 382 and the patient entered data 383.

The contextual data 384 may include any kind of information that may provide more context to the sensor data 382 and/or the patient entered data 383. For example, the contextual data 384 may include data from the biofluids monitoring data source 340. As another example, contextual data may include geographical information about the patient (e.g., which region of the country the patient resides), information about the disease condition of a cohort similar to the patient. Generally, any type of information that generates additional healthcare data points within the operating environment 300 should be considered as contextual data 384. As with the sensor data 382 and the patient entered data 383, the contextual data 384 may also be timestamped, such that these three types of data may be temporally correlated during further analysis (e.g., by side effects predictor 350).

The historical event logs 385 may include a record of events associated with the patient. For instance, the historical event logs 385 may include other information on clinical encounters, prescription filling and refilling, and/or any other types of events associated with managing side effects of oncology treatment regimen. The historic event logs 385 may also be timestamped such that these logs can be temporally correlated with one or more of the sensor data 382, patient entered data 383, or contextual data 384.

The analytic components (e.g., side effects predictor 350) may use the individual records 380 in the storage 370 (and/or other types of data) to generate/train one or more machine learning models (and/or any other type of analytic models), and then deploy the trained models for one or more of predicting an onset of side effects such as cytopenia.

The side effects predictor 350 may predict a likelihood of a side effects for a patient undergoing oncology treatment regimen. Such prediction may be based on using a prediction model 352, which may be trained by a model trainer 354 and deployed by a model deployer 356. The prediction model 352 may include, for example, regression model, a gradient boosted regression model, a logistic regression model, a random forest regression model, an ensemble model, a classification model, a deep learning neural network, a recurrent neural network for deep learning, or a convolutional neural network for deep learning.

The model trainer 354 may train the prediction model using a training dataset. To that end, the model trainer 354 may include computer program instructions that may retrieve the training data set, pre-process the training data set, and use the training data to train the prediction model 352, e.g., using one or more of supervised or unsupervised training approaches. In an example training using a supervised training approach, the model trainer 354 may retrieve a labeled training dataset. The labeling may have been done by humans based on past observations. For instance, the labeled dataset may have a set of healthcare data inputs, such as data collected passively by wearable sensors, invisible sensors, etc., collected with active patient engagement on the applications running the patient user devices, and/or data of laboratory results (e.g., bloodwork). These inputs may be associated with observed outputs—for example, if a patient associated with a first set of inputs developed a side effect, that data may be human labeled as being associated with the side effect. Similarly, if a patient associated with a second set of inputs did not develop the side effect, the corresponding data may be human labeled as not being associated with the side effect.

Therefore, using the labeled dataset (e.g., where the ground truth of the output is provided with the inputs), the model trainer 354 may train the prediction model 352. However, it should be understood that the supervised training approach is just but one approach and should not be considered limiting. The model trainer 354 may use other approaches, e.g., unsupervised training approach to train the prediction model 352.

Although the prediction model 352 is described herein as a machine learning model, it should be understood that the prediction model 352 may include a statistical model. For the statistical model, the model trainer 354 may function as a model generator to generate the statistical model. The statistical model may be used for predicting which combinations of input variables (e.g., healthcare data from various sources) may more likely result in a “cytopenia” output; and which combinations of the input variables may more likely result in a “no-cytopenia” output.

It should be understood that the model trainer 354 may continuously train the prediction model 352. For instance, if the ground truth is available for a prediction (e.g., the ground truth may indicate whether the prediction was correct or incorrect), the model trainer 354 may use such correct or incorrect prediction to continuously train and improve upon the prediction model 352.

The model deployer 356 may be software module using the trained prediction model 352 to predict the likelihood of a side effect (e.g., cytopenia) from received input data. For example, a new healthcare data may be received for a patient undergoing the oncology treatment regimen (e.g., CDK inhibitor treatment regiment, PARP inhibitor treatment regimen). The new healthcare data may include, for example, passively collected data from wearable device 302a, data collected with patient engagement from the patient user device 302b, data collected from other sensors 302c, data collected from the biofluids monitoring data source 340, etc. The healthcare data may include, for example, heart rate, skin temperature, blood pressure, blood oxygen saturation, etc. When this healthcare data is fed into the trained prediction model 352, the prediction model may output a likelihood of a side effect. The likelihood may be expressed as probabilistic output, e.g., 85% likely to develop cytopenia and 15% not likely to develop cytopenia. Alternatively or additionally, the likelihood may be expressed as a binary classification, e.g., “likely to develop cytopenia” and “not likely to develop cytopenia.” The binary classification may be driven by the underlying probabilistic output and may use a threshold. For instance, the binary classification may output “likely to develop cytopenia” when the probabilistic output crosses “55% likely to develop cytopenia” threshold. The threshold may be adjusted as the prediction model 352 is continuously trained and refined.

The communication facilitator 358 may generate one or more notifications based on the trained prediction model 352 indicating a higher likelihood of a side effect. A notification may be provided to the clinician dashboard 342. In some instances, the notification may provide the probabilistic likelihood of the patient developing the side effect. In other instances, the notification may be binary output of whether or not the patient is likely to develop the side effect. The dashboard application 342 may be showing a view of the patient's profile/health data 381 and the notification may be presented in the view. In addition to a notification to the clinician dashboard 342, the communication facilitator 358 may transmit another notification to the healthcare application 332 running on the patient user device 302b. The notification to the patient may not necessarily indicate a likelihood of the side effect, but simply indicate that a clinician will be following up on healthcare matter. One or more of these notifications may also establish a two-way connectivity between the patient and the clinician, either through the healthcare application 332 itself or through other components within the operating environment 300.

For example, of the prediction model 362 determines that the patient may likely develop a side effect (e.g., cytopenia), the communication facilitator 358 may transmit a first alert notification to the patient (e.g., to be displayed on the healthcare application 332 of the patient user device 302b) and a second alert notification to the clinician (e.g., to be displayed on the dashboard application 342 of the clinician user device 308). One or more of these alerts may have a communication prompt. For instance, the first alert to the patient may have a prompt “Send A Message To My Doctor.” The second alert to the clinician may be “Reach Out to Patient A, She May Be Developing Cytopenia.” In response to these prompts, an asynchronous (e.g., through text message exchange) or synchronous (e.g., through audio/video chat) communication channel may be opened between the healthcare application 332 and the dashboard application 342. In addition to this two-way connectivity, the clinician may be able to perform other actions such as prescribe medications, provide educational materials, and/or any perform any other type of patient care actions. As described above, the two-way connectivity does not have to be necessarily between the healthcare application 332 and the dashboard application 342 and can be provided by any other type of component (e.g., a telephone call).

Based on the output of the prediction model 352 and the subsequent notification to the clinician dashboard application 342, the clinician may determine clinical intervention such as prescribing medication, recommending the patient to change diet or activity level, etc. Generally, the output of the prediction model 352 may provide clinical decision support.

In some embodiments, the model trainer 354 may generate models (e.g., train the prediction model 352 or generate an analytical model) for individual patients. For instance, the model trainer 354 may retrieve long-term data for individual patients and then establish a baseline as a corresponding trained prediction model 352. The baseline may include, for example, normal levels of physical activity, biological data (e.g., blood oxygen saturation level, etc.), the symptoms reported by the patient (e.g., “feeling fatigued”), and/or biofuel analysis data such as bloodwork data (e.g., normal white blood cell count). The combination of the normal levels of these attributes may therefore be established as a baseline in the trained prediction model 352 or the generated analytical model. In the deployment phase, the newly received healthcare data may be compared against the baseline to determine whether there is significant amount of deviation from the established baselines.

In some embodiments, the model trainer 354 may generate population level baselines. For instance, the model trainer 354 may retrieve long-term data for a population of patients with certain criteria, e.g., age, gender, geographical location, ethnicity, etc. Based on the collected data, the model trainer 354 may establish a population level baseline in the trained prediction model 352 (and/or a statistical model). The model deployer 346 may later use the population level baseline to determine whether an individual patient's condition has deviated significantly from the normal level, and therefore the patient may have been likely to develop a side effect.

The dashboard application 342 may be displayed on a clinician user device 308, which may be any kind of user device used by a clinician. Non-limiting examples of the clinician user device 308 may include mobile phone (e.g., smartphone), tablet computer, laptop computer, desktop computer, etc. The dashboard application 342 may be an installed stand-alone application or web-based application accessible through a browser. The dashboard application 342 may show the disease progress of an individual patient. For instance, the dashboard application 342 may show a chart showing how the biological data (e.g., blood oxygen saturation level) has changed over time. The dashboard application may also other aspects of the patient's health, e.g., levels of stress and anxiety, etc. The dashboard application 342 may further show the medications prescribed for the patient. Generally, the dashboard application 342 may show any type of clinical data and notifications for the patient being treated and monitored in in the operating environment 300.

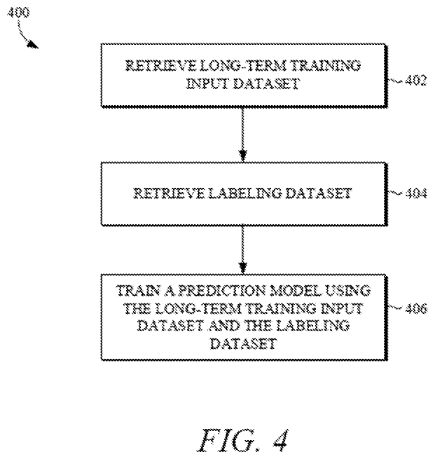

FIG. 4 shows a flow diagram of an illustrative method 400 of training a prediction model for predicting side effects of patients undergoing CDK treatment regimens, according to several embodiments of this disclosure. The illustrative method 400 may be implemented by one or more computing devices (e.g., computing device 200 as used in the operating environment 100). It should be understood that the steps of the method 400 shown in FIG. 4 and described herein are merely illustrative and methods with additional, alternative, or a fewer number of steps should be considered within the scope of this disclosure.

At step 402, long-term training input dataset may be retrieved. The long-term training dataset may include healthcare data for a population of patients that may have undergone oncology treatments (e.g., CDK inhibitor treatments, PARP inhibitor treatments, etc.). The healthcare data may include, for instance, data collected through patient engagement in applications (prescription software) installed on patient devices, passively collected biological data (e.g., temperature, blood oxygen saturation level, heartrate, blood pressure) from wearables, data from integrated biofluid test systems (e.g., at-home test kits) for the patients undergoing oncology treatments. In addition, the long-term training input dataset may include other information such as the patients' demographic information, medical history, family medical history, and/or any other healthcare attribute that may likely influence the oncology treatment. The dataset retrieved at this step may be used as inputs for training the prediction model.

At step 404, a labeling dataset may be retrieved. The labeling dataset may generally indicate whether patients associated with the long-term training input dataset actually developed a side effects related to the oncology treatments. The labeling dataset may therefore come from various sources. For instance, the labeling dataset may be sourced from the patients' electronic health records (e.g., EHR 381 shown in FIG. 3). In some embodiments, the labeling dataset retrieved at step 404 may be used to manually label the long-term training input dataset retrieved at step 402.

Therefore, the combination of the long-term training input dataset and the labeling dataset may provide labeled training dataset for training a prediction model in step 406. Using the labeled training dataset, the prediction model may be trained with a supervised training approach. For instance, each training iteration may generate an output, which may be compared against the expected output (e.g., as shown by the labels), and backpropagation techniques may be used to refine the prediction model such that the prediction model generates an output closer to the expected output. Some non-limiting examples of the prediction model may include a regression model, a gradient boosted regression model, a logistic regression model, a random forest regression model, an ensemble model, a classification model, a deep learning neural network, a recurrent neural network for deep learning, or a convolutional neural network for deep learning.

This approach of training the prediction model is just an example and other approaches should also be considered within the scope of this disclosure. For example, the prediction model may be trained using an unsupervised training approach. In other examples, the prediction model may be a statistical model, and step 406 may establish the statistical model using the retrieved datasets. Therefore, any type of data analytics to generate a prediction model or to establish a statistical model should be considered within the scope of this disclosure.

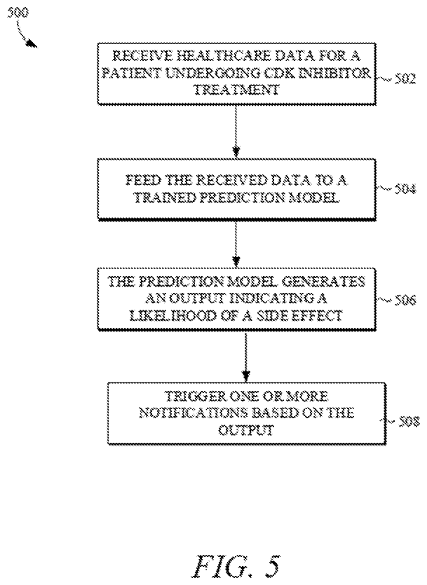

FIG. 5 shows a flow diagram of an illustrative method 500 of using a trained prediction model for predicting side effects for patients undergoing oncology treatments (e.g., CDK inhibitor treatments, PARP inhibitor treatments, etc.), according to some embodiments of this disclosure. The illustrative method 500 may be implemented by one or more computing devices (e.g., computing device 200 as used in the operating environment 100). It should be understood that the steps of the method 500 shown in FIG. 5 and described herein are merely illustrative and methods with additional, alternative, or a fewer number of steps should be considered within the scope of this disclosure.

At step 502, healthcare data for a patient undergoing oncology treatment may be received. As a companion to the oncology treatment, the patient may be provided with a prescription software, e.g., a healthcare application installed in a smartphone. The patient may enter symptoms and/or medically relevant data on the healthcare application. The patient may also be provided with a wearable device (e.g., a smartwatch). The wearable device may passively capture data such as movement, blood oxygen saturation level, blood pressure, heart rate, body temperature, etc. Additionally, the healthcare data may include biofluid analysis data from integrated biofluid testing systems such as at-home biofluid testing kits and laboratory testing systems. Therefore, the healthcare data from the multiple sources may be used as inputs to a trained prediction model.

At step 504, the received data may be fed to the trained prediction model. The prediction model may have been trained using the steps of the method 400. The trained prediction model should be understood also to include any type of established statistical model. Some non-limiting examples of the prediction model may include a regression model, a gradient boosted regression model, a logistic regression model, a random forest regression model, an ensemble model, a classification model, a deep learning neural network, a recurrent neural network for deep learning, or a convolutional neural network for deep learning. In some cases, a statistical model may be used, where step 504 may include comparing a statistic (e.g., a z-statistic) to determine if the received data is significantly closer a likely outcome (e.g., an outcome indicating a side effect such as cytopenia). It should however be understood that these are just examples and other prediction/statistical model should be considered within the scope of this disclosure.

At step 506, the prediction model (or a statistical model) generates an output indicating a likelihood of a side effect, such as cytopenia. The likelihood of the side effect may indicate corresponding of probabilities of the fed inputs being associated with the side effect and not being associated with the side effect. For example, the output may be probability of a cytopenia 90% and probability of no-cytopenia 10%. In other embodiments, the outputs may be binary: for example, if the probabilistic likelihood crosses a certain threshold, the output may be likely cytopenia, and not likely cytopenia otherwise.

At step 508, one or more notifications may be triggered based on the output of the prediction model. For example, if the prediction model generates a higher likelihood of cytopenia, a notification may be triggered to the clinician's dashboard. The notification may indicate that a certain patient may likely develop cytopenia. This notification may provide a clinical decision support to the clinician for in planning a clinical intervention. For instance, the clinician may prescribe a medication for patients with a higher likelihood of cytopenia in combination with a lower dosage of oncology treatment (e.g., a lower dosage of a CDK inhibitor). For patients with some likelihood of cytopenia, the clinician may recommend diet and lifestyle changes without necessarily lowering the dosage of the oncology treatment. In addition to the notification to the clinician, notification to the patients may also be generated. A notification to the patient may include that the clinician will reach out the patient with important clinical information.

It will be appreciated by those skilled in the art that the present disclosure can be embodied in other specific forms without departing from the spirit or essential characteristics thereof. The presently disclosed embodiments are therefore considered in all respects to be illustrative and not restricted. The scope of the disclosure is indicated by the appended claims rather than the foregoing description and all changes that come within the meaning and range and equivalence thereof are intended to be embraced therein.

It should be noted that the terms “including” and “comprising” should be interpreted as meaning “including, but not limited to”. If not already set forth explicitly in the claims, the term “a” should be interpreted as “at least one” and “the”, “said”, etc. should be interpreted as “the at least one”, “said at least one”, etc. Furthermore, it is the Applicant's intent that only claims that include the express language “means for” or “step for” be interpreted under 35 U.S.C. 112(f). Claims that do not expressly include the phrase “means for” or “step for” are not to be interpreted under 35 U.S.C. 112(f).

1. A computer-implemented method comprising:
retrieving first health data comprising symptoms of a patient undergoing a cyclin-dependent kinase (CDK) inhibitor treatment;
retrieving second health data comprising analysis of a biofluid sample collected from the patient;
deploying a machine learning model on the first health data and the second health data to predict whether the patient will develop a side effect associated with the CDK inhibitor treatment; and
in response to the machine learning model predicting that the patient will likely develop a side effect, generating a message to be transmitted to a clinician dashboard to trigger a notification on the clinician dashboard.

2. The computer-implemented method of claim 1, further comprising:
training the machine learning model using a supervised approach by passing through labeled data of a cohort of patients through the model.

3. The computer-implemented method of claim 1, wherein the machine learning model comprises at least one of a regression model, a gradient boosted regression model, a logistic regression model, a random forest regression model, an ensemble model, a classification model, a deep learning neural network, a recurrent neural network for deep learning, or a convolutional neural network for deep learning.

4. The computer-implemented method of claim 1, wherein the first health data is based on the patient's active entry of the symptoms on a healthcare application executing on a client device associated with the patient.

5. The computer-implemented method of claim 1, wherein the first health data based on data collective passively by a wearable device worn by the patient.

6. The computer-implemented method of claim 1, wherein the second health data is retrieved from an at-home biofluid collection kit.

7. The computer-implemented method of claim 1, wherein the second health data is retrieved from a laboratory system.

8. The computer-implemented method of claim 1, wherein triggering the notification on the clinician dashboard comprises providing the notification to an electronic health record (EHR) system.

9. The computer-implemented method of claim 1, wherein the message to be transmitted to the clinician dashboard comprises at least one of an indication that the clinician should contact the patient, an indication that dosage of a prescription medication is to be adjusted, or indication that the patient should be admitted to the hospital.

10. A computer-implemented method comprising:
retrieving first health data comprising symptoms of a patient undergoing a cyclin-dependent kinase (CDK) inhibitor treatment;
retrieving second health data comprising analysis of a biofluid sample collected from the patient;
deploying a machine learning model on the first health data and the second health data to predict whether the patient will develop a side effect associated with the CDK inhibitor treatment; and
in response to the machine learning model predicting that the patient will likely develop a side effect, generating a message to be transmitted to a healthcare application executing on a client device associated with the patient to trigger a notification on the healthcare application.

11. The computer implemented method of claim 10, further comprising:
training the machine learning model using a supervised approach by passing through labeled data of a cohort of patients through the model.

12. The computer implemented method of claim 10, wherein the machine learning model comprises at least one of a regression model, a gradient boosted regression model, a logistic regression model, a random forest regression model, an ensemble model, a classification model, a deep learning neural network, a recurrent neural network for deep learning, or a convolutional neural network for deep learning.

13. The computer-implemented method of claim 10, wherein the message to be transmitted comprises at least one of an indication that the patient should contract the clinician, an indication that the patient should pick up medication at the pharmacy, or an indication that the patient should contact emergency services.

14. The computer-implemented method of claim 10, further comprising:
establishing, based on the notification on the healthcare application, two-way connectivity between the patient and a clinician.

15. The computer-implemented method of claim 10, wherein the side effect comprises cytopenia.

16. The computer-implemented method of claim 10, further comprising:
prompting the patient to enter the symptoms on the healthcare application executing on the client device associated with the patient; and
retrieving the symptoms as the first health data from the healthcare application.

17. The computer-implemented method of claim 10, further comprising:
triggering a wearable device worn by the patient to passively collect biological data of the patient; and
retrieving the biological data passively collected by the wearable device as the first health data.

18. A system comprising:
one or more processors; and
a non-transitory storage medium storing computer program instructions that when executed by the one or more processors cause the system to perform operations comprising:
retrieving first health data comprising symptoms of a patient undergoing a cyclin-dependent kinase (CDK) inhibitor treatment;
retrieving second health data comprising analysis of a biofluid sample collected from the patient;
deploying a machine learning model on the first health data and the second health data to predict whether the patient will develop a side effect associated with the CDK inhibitor treatment; and
in response to the machine learning model predicting that the patient will likely develop a side effect, triggering one or more notifications.

19. The system of claim 18, wherein the one or more notifications comprise at least one patient notification on a healthcare application executing on a client device associated with the patient or a notification on a clinician dashboard.

20. The system of claim 18, where in the operations further comprise:
training the machine learning model using a supervised approach by passing through labeled data of a cohort of patients through the model,
wherein the machine learning model comprises at least one of a regression model, a gradient boosted regression model, a logistic regression model, a random forest regression model, an ensemble model, a classification model, a deep learning neural network, a recurrent neural network for deep learning, or a convolutional neural network for deep learning.
